(19) United States
(12) Patent Application Publication
(10) Pub. No.: US 2026/0185123 A1
(43) Pub. Date: Jul. 2, 2026

(54) METHOD FOR PREPARING A SUBSTRATE FOR FERMENTATION

(71) Applicant: INTACT, Olivet (FR)

(72) Inventors: Alexis DUVAL, Olivet (FR); Christopher HERVÉ, Olivet (FR); Didier LEFEBVRE, Olivet (FR); Cédric LAUNAY, Olivet (FR)

(21) Appl. No.: 19/129,918

(22) PCT Filed: Nov. 11, 2023
(86) PCT No.: PCT/FR2023/051775

§ 371 Date: May 14, 2025

(30) Foreign Application Priority Data
Nov. 15, 2022 (FR) FR2211884

(51) Int. Cl. C12P 7/10

(52) U.S. Cl. CPC C12P 7/10

(57) ABSTRACT

The invention relates to a method for preparing a fermentation substrate, said method comprising the steps of: providing at least one leguminous plant seed comprising starch and proteins; micronizing the seed so as to obtain the micronized fraction; purifying the micronized fraction so as to collect a starch-enriched and protein-depleted fraction; mixing the starch-enriched and protein-depleted fraction with a liquid so as to form a starch fluid; and hydrolysing the starch by mixing water vapour with the starch fluid so as to obtain a hydrolysed starch fluid. The invention relates to a fermentation method, and to a fermentation product.

### FIELD OF THE INVENTION

The present invention relates to methods for preparing a substrate usable in fermentation from a leguminous plant that emit low greenhouse gas emissions and fermentation substrates obtained by such methods.

### TECHNICAL BACKGROUND

The fermentation industry, such as the production of ethanol, organic acids, amino acids, or vitamins, requires substrates rich in glucose or sucrose. Glucose is the basic food for the microorganisms necessary for the production of the aforementioned products.

To obtain this glucose, the raw material used is mainly a cereal rich in carbohydrates such as wheat and corn or, when the substrate contains sucrose, beet or sugarcane. When the raw material contains sucrose, it is traditionally extracted from the plant to obtain the sugar juice that will serve as a substrate for fermentation. When the raw material contains starch, the typical glucose production method comprises a first step of producing flour from this raw material, followed by a liquefaction (or hydrolysis) step to obtain a starch-rich milk, followed by saccharification to convert the starch into fermentable glucose that will serve as an ingredient in a fermentation substrate.

More specifically, when wheat is used as a source of glucose for a method for ethanol production, the grain is first ground to separate the bran from the flour. The flour is then diluted in water in order to reach about 30% dry matter, and an enzyme such as α-amylase is added to this mixture. The milk, thus obtained, is introduced into a tank containing steam injection rods. By steam injection, the temperature of the milk will increase to about 95° C., so that the starch granules contained in the flour burst and release the starch chains into the medium. Starch is a glucose polymer composed of a mixture of two homopolymers: amylose, which is a linear glucose polymer in which the glucose units are linked by α(1→4) bonds, and amylopectin, which is a glucose polymer in a so-called “branched” form, in which the presence of α(1→6) bonds (in addition to α(1→4) bonds) causes numerous branches. The presence of α-amylase will allow the hydrolysis of these chains into smaller fragments called dextrin. The size of the tank is generally dimensioned to obtain a residence time of 1 to 2 hours, so as to allow sufficient contact time for the enzyme to sufficiently degrade the starch chains into dextrin. According to a first variant of the method, the dextrin solution then obtained is cooled to 60° C. before being transferred to another tank. Another enzyme such as glucoamylase is added to hydrolyze the dextrin into glucose. This operation, called pre-saccharification, typically takes between 3 and 4 hours depending on the enzyme dosage, and allows a minimum glucose level necessary for the growth of microorganisms to be obtained. The glucose solution thus obtained is cooled again to reach a temperature of about 30° C. and is then transferred to a so-called fermentation tank. In the case of ethanol production, yeasts are added to the tank. These microorganisms will consume the glucose and produce ethanol, intended, in particular, for the food, pharmaceutical, and green chemistry industries.

According to a second variant of the method, no pre-saccharification is performed but a propagation step. The dextrin solution obtained after liquefaction is directly cooled to 30° C. to be injected into a tank. At the same time, the yeast and the glucoamylase are injected. The temperature being at 30° C., the glucoamylase has a reduced activity but sufficient activity to provide the glucose necessary for yeast growth.

In cases where glucose from wheat is used in the production of organic acids, vitamins, or amino acids, the method for obtaining glucose is generally different. After the liquefaction step, the saccharification of dextrin must be complete, unlike the method used for ethanol production. The saccharification time is comprised between 40 and 60 hours so that all the dextrin has been converted into glucose. At the end of saccharification, the glucose content must be greater than 95% on dry matter. For these productions of organic acids, vitamins, or amino acids, the glucose solution must be free of all insoluble species and contain the lowest possible level of soluble species other than glucose (such as proteins, fats, minerals, and other organic matter) to be used as a fermentation substrate. Purification steps are therefore usually performed on the glucose solution, such as filtration, ion exchange, and adsorption steps.

When the raw material is beet, the method for sugar extraction begins with washing the root to remove the rootlets, then the washed beet is cut into small sticks called cossettes. These are introduced into a device called a diffuser, containing a volume of water that circulates counter-current and that is heated to 80° C. During this operation, the soluble compounds of the beet migrate into the water by a process of osmosis. The water, enriched with soluble compounds (the “diffusion juice”), exits at the head of the diffuser, and the cossettes, depleted of their sugar, exit at the rear of the diffuser in the form of pulp. The diffusion juices are then subjected to treatment by lime (or liming) in order to precipitate part of the impurities present in the juice. Liming is generally followed by double carbonation (addition of CO₂) to precipitate the remaining lime in the juice. The impurities and precipitated lime are then separated from the juice by filtration. The purified juice is then subjected to an evaporation step making it possible to concentrate it until a syrup, having a sugar concentration close to saturation, is obtained. Evaporation typically takes place in a “multiple effect” evaporator (several successive evaporators) in which the pressure is lowered from effect to effect to reduce the boiling point of the concentrated juice. The syrup obtained can then be used in a fermentation method that is most often intended for ethanol production.

The methods described above emit very large quantities of greenhouse gas, and notably carbon dioxide and nitrous oxide, due to the methods described previously and the plants used. Indeed, these plants need to find significant amounts of mineral nitrogen in the soil to grow (2 to 3 kg for 100 kg of corn or wheat). This nitrogen is then provided by nitrogen fertilizers, which constitute the main cause of greenhouse gas emissions related to the cultivation of these plants due to the significant emissions involved in the production of synthetic nitrogen (Haber-Bosch process) and the volatilization of nitrous oxide resulting from the application of fertilizers.

There is therefore a real need to develop a method for preparing a substrate usable in fermentation, notably for the production of ethanol or other organic compounds on an industrial scale, with reduced greenhouse gas emissions.

### SUMMARY OF THE INVENTION

The invention relates primarily to a method for preparing a fermentation substrate, comprising the following steps:
providing at least one leguminous plant seed comprising starch and proteins;
micronizing said at least one seed in such a way as to obtain a micronized fraction;
purifying the micronized fraction in such a way as to collect a fraction enriched in starch and depleted in protein;
mixing the fraction enriched in starch and depleted in protein with a liquid in such a way as to form a starch fluid; and
hydrolyzing the starch by mixing steam with the starch fluid in such a way as to obtain a hydrolyzed starch fluid.

In some embodiments, the method further comprises a step of introducing, into the hydrolyzed starch fluid, at least one enzyme chosen from the group consisting of glucosidases, preferably at least two enzymes chosen from the group consisting of glucosidases, more preferably at least one α-1,4-glucosidase and one amylo-α-1,6-glucosidase.

In some embodiments, the method further comprises a step of introducing, into the starch fluid, at least one enzyme chosen from the group consisting of saccharidases, preferably an α-amylase.

In some embodiments, the leguminous plant seed comprises a hull, and the method comprises a step of dehulling the seed prior to the micronization.

In some embodiments, the hydrolysis of the starch is performed using a device for direct steam injection in continuous mode.

In some embodiments, the purification of the micronized fraction comprises an aeraulic separation step, preferably by means of a cyclone with a selector.

In some embodiments, the leguminous plant is chosen from the group of beans, peas, broad beans, lentils, chickpeas and mixtures thereof.

In some embodiments, the method generates an emission of greenhouse gas less than 100, preferably 60, kg CO₂ oil equivalent per ton of leguminous plant seed used.

The invention also relates to a fermentation substrate obtained by a method as described above.

The invention also relates to a method of fermentation comprising bringing into contact a fermentation substrate as described above with at least one microorganism.

The invention also relates to a method for producing a fermentation product, comprising the following steps:
preparing a fermentation substrate according to a method as described above; and
bringing said fermentation substrate into contact with at least one microorganism to obtain a fermentation product.

In some embodiments, the fermentation product produced by the above method comprises at least one compound chosen from the group consisting of alcohols, preferably ethanol, organic acids, amino acids, vitamins and mixtures thereof.

In some embodiments, the microorganism is chosen from the group consisting of yeasts, bacteria and combinations thereof.

The invention also relates to a fermentation product obtained by a method as described above.

In some embodiments, the fermentation product comprises at least one compound chosen from the group consisting of alcohols, preferably ethanol, organic acids, amino acids, vitamins and mixtures thereof.

The present invention allows the need expressed above to be addressed. It provides more particularly a method for preparing a fermentation substrate that is more environmentally friendly, and more particularly with low greenhouse gas emissions, while allowing the production of a nutrient-rich substrate for fermentation, in particular rich in glucose, and being able to be produced on an industrial scale.

This is accomplished by using a specific raw material, namely a leguminous plant, and by combining a micronization step of said leguminous plant with a purification step of the micronized leguminous plant allowing a fraction enriched in starch and depleted in protein to be obtained.

### BRIEF DESCRIPTION OF THE FIGURES

FIG. 1 represents a schematic representation of an example of a device for direct steam injection in continuous mode, usable in the invention. The arrows represent the flow direction of the streams.

### DETAILED DESCRIPTION

The invention is now described in more detail and in a non-limiting manner in the following description.

Unless otherwise indicated, all percentages are by mass percentages.

In the present text, the quantities indicated for a given species may apply to that species according to all its definitions (as mentioned in the present text), including more restricted definitions.

The invention relates to a method for preparing a substrate from at least one leguminous plant seed.

By “leguminous plant”, we mean plants of the Fabaceae family. Leguminous plant seeds comprise, in particular, starch and proteins. They have the advantage of being rich in carbohydrates (they can comprise about 60% carbohydrate), mainly in the form of starch.

All leguminous plants are suitable for the invention. As examples of leguminous plants usable in the invention, mention may be made of beans, peas, broad beans, lentils, chickpeas and mixtures thereof.

The use of leguminous plants as a raw material is advantageous because their cultivation results in low greenhouse gas emissions. Indeed, leguminous plants are the only plants capable of fixing nitrogen from the air into the soil, through their symbiotic association with bacteria of the genus Rhizobium via the formation of nodules, which allows the plant to be provided with the nitrogen necessary for its growth. The ability of leguminous plants to fix nitrogen from the air allows the use of nitrogen fertilizers to be avoided, which, when applied in excess, harm soil biodiversity and thus their fertility. Moreover, the application of nitrogen fertilizers releases a large amount of nitrous oxide, which is a greenhouse gas. Furthermore, the nitrogen from the air fixed by leguminous plants is returned to the next crop via the decomposition of crop residues (aerial and underground parts) by the Rhizobium bacteria, with the most easily degradable residues (leaves, non-woody stems with a low carbon/nitrogen ratio) decomposing and releasing nitrogen in a few weeks, while the woody parts (stems, roots) mineralize more slowly. More particularly, the carbon emissions related to leguminous plant cultivation are estimated at 200 kg CO₂ oil equivalent per ton of leguminous plants. When leguminous plant cultivation is associated with cereal crops in a crop rotation process (for example, alternating pea, wheat, and oat crops), the input of nitrogen fertilizers is reduced, which can allow carbon emissions to be reduced by 189 kg CO₂ oil equivalent per ton of leguminous plant: leguminous plant cultivation in this case therefore has a nearly neutral net balance of 11 kg CO₂ oil equivalent per ton of leguminous plant.

Leguminous plant seeds are a plant material comprising a hull and a kernel. However, in the present text, the term “seed” may generally refer to the whole seed as well as any part of the seed (for example, the kernel), unless otherwise indicated.

Preferably, a leguminous plant seed comprising a hull and a kernel is used as the starting raw material.

Advantageously, the method according to the invention comprises a step of removing the hull (or dehulling) of the leguminous plant seeds. Indeed, the hull is mainly composed of insoluble fibers that are not consumed by fermentation microorganisms. Moreover, most of the contaminants of the leguminous plant seed are found in the hull, so its removal thus allows the risk of contamination of the prepared substrate to be reduced. Furthermore, the presence of fibers also increases the viscosity of the prepared fermentation substrate, which limits the dry matter content of this substrate. The mass quantity of fibers can, for example, represent 8 to 10% of the total dry matter of the leguminous plant seed.

Preferably, dehulling is mechanical, performed, for example, by abrasion, compression, impact, shearing, or any other appropriate mechanical action.

Advantageously, dehulling is performed by grinding the seed and then separating the obtained particles based on their size. For grinding, any suitable grinder can be used, notably any grinder using one of the mechanical forces mentioned above. In particular, a pendular mill using compression force can be used. After grinding, a mixture of hull fragments and kernel powder (also called flour in the present text) is obtained.

The step of separating the obtained particles is preferably performed by sieving. In particular, the particles can be separated by passing them over a sieve with an appropriate mesh size allowing the hull fragments to be separated from the flour. For example, particles smaller than a size between 200 and 600 mn can be separated from hull fragments of a size greater than or equal to such a size.

The leguminous plant seed, preferably the dehulled seed, more preferably the flour, is subjected to a step of grinding, and a ground fraction is collected.

In a particularly advantageous manner, grinding comprises, or is, micronization, preferably dry micronization. The ground fraction obtained is then a micronized fraction.

The leguminous plants have a protein content in dry matter much higher than plants traditionally used as raw materials in fermentation industries (cereals, beets, and sugarcane). The protein content of leguminous plants can, for example, reach up to about 30% by weight in dry matter compared to about 10 to 12% by weight for cereals and even considerably less for beet and sugarcane. Such a high protein content causes certain difficulties in traditional preparation methods. First, during the step of starch hydrolysis, the applied temperature causes the coagulation of proteins, which then become insoluble. A significant amount of insoluble proteins in combination with the presence of fiber can lead to the formation of a magma that is difficult to transfer from one step to another and poses a risk of creating a blockage in the pipes. Furthermore, the formation of a magma restricts the technologies usable during the hydrolysis step, complicating or preventing the use of certain devices, particularly devices for direct steam injection in continuous mode. Indeed, in such devices, the formation of a protein and fiber blockage can lead to a very significant increase in pressure in the device and cause damage to the device (particularly the explosion of seals), or even its explosion, which can also injure an operator nearby. In addition, a significant amount of protein also leads to a high level of free amino acids. During fermentation, this high level of free amino acids can promote the growth of undesirable microorganisms such as acetic bacteria instead of yeasts in the case of ethanol production.

The micronization step in combination with the subsequent purification step allows to overcome the disadvantages mentioned above caused by a significant amount of protein. The step of micronization allows the proteins to be disassociated from the starch granules to allow their subsequent separation.

By “micronization”, is meant grinding that allows to obtain particles with a median volume diameter of less than 100 μm, preferably less than 50 μm, more preferably less than 30 μm. The median volume diameter (D50) of the particles can be measured according to NF ISO 13320-1.

Micronization can be performed by any suitable grinder (notably any grinder using mechanical forces of abrasion, compression, impact, or shearing). For example, a grinder using impact force can be used.

Micronization is most preferably performed at room temperature (that is, between 15 and 30° C.).

Micronization presents the additional advantage of being low in greenhouse gas emissions, as it is preferably performed at room temperature and by dry processes.

The method according to the invention comprises a step of purification of the micronized fraction. Advantageously, this purification comprises a step of separating the starch granules from the proteins.

At the end of purification, a fraction enriched in starch and depleted in protein is collected. A fraction enriched in protein and depleted in starch is also preferably recovered. By “fraction enriched in starch and depleted in protein”, is meant a fraction in which the ratio of molar proportions of starch/protein (on dry matter) is greater than that of the fraction subjected to purification. By “fraction enriched in protein and depleted in starch”, is meant a fraction in which the ratio of molar proportions of starch/protein (on dry matter) is less than that of the fraction subjected to purification.

Given the size difference between proteins and starch granules (D50 of about 1 to 5 μm for proteins and about 10 to 30 μm for starch granules) and their density difference, a separation based on a difference in size, density, or weight of the particles can advantageously be used. Preferably, the separation is an aeraulic separation. By “aeraulic separation”, is meant any separation technology using a jet of gas (preferably air) carrying at least part of the particles along. More preferably, the separation is a cyclonic separation. It can be performed by means of a cyclone, advantageously associated with a selector. By “selector”, is meant any variable-speed rotary element equipped with radial blades installed in a part (preferably the upper part) of a cyclonic separator. This equipment makes it possible to increase the efficiency of particle separation based on their density. The use of an aeraulic separation device allows the recovery of the lightest particles, carried along by the gas flow, at one end of the device (fraction enriched in protein and depleted in starch), while the heavier particles are collected at another end (fraction enriched in starch and depleted in protein).

Preferably, the fraction enriched in starch and depleted in protein comprises an amount of carbohydrate greater than or equal to 40% by weight, preferably an amount of 40 to 90% by weight, more preferably 50 to 80% by weight, more preferably 60 to 80% by weight (relative to the total dry matter weight of the fraction). In particular, the amount of carbohydrate in the recovered fraction enriched in starch and depleted in protein can comprise 40 to 50% by weight, or 50 to 60% by weight, or 60 to 65% by weight, or 65 to 70% by weight, or 70 to 75% by weight, or 75 to 80% by weight, or 80 to 90% by weight, relative to the total dry matter weight of the fraction.

Preferably, the fraction enriched in starch and depleted in protein comprises an amount of protein less than or equal to 30% by weight, preferably an amount of 0.5 to 30% by weight, more preferably 3 to 20% by weight, more preferably 5 to 15% by weight (relative to the total dry matter weight of the fraction). In some embodiments, the amount of protein in the recovered fraction enriched in starch and depleted in protein can be, relative to the total dry matter weight of the fraction, 0.5 to 3% by weight, 3 to 5% by weight, or 5 to 7% by weight, or 7 to 10% by weight, or 10 to 12% by weight, or 12 to 15% by weight, or 15 to 20% by weight, or 20 to 30% by weight.

Preferably, the fraction enriched in starch and depleted in protein comprises an amount of fiber less than or equal to 10% by weight, preferably an amount of 0.5 to 10% by weight, more preferably 1 to 6% by weight, relative to the total dry matter weight of the fraction; in particular, the fraction can comprise an amount of fiber of 0.5 to 2% by weight, or 2 to 4% by weight, or 4 to 6% by weight, or 6 to 8% by weight, or 8 to 10% by weight, relative to the total dry matter weight of the fraction. By “fiber” is meant all plant polymeric molecules, soluble or insoluble, other than starch and starch fragments. Fibers include, in particular, cellulose, hemicellulose, lignin, β-glucans, and pectin.

Preferably, the collected starch fraction comprises an amount of fat (lipid) less than or equal to 5% by weight, preferably an amount of 0.5 to 5% by weight, more preferably 0.5 to 3% by weight, relative to the total dry matter weight of the fraction; in particular, the fraction can comprise an amount of fat of 0.5 to 1% by weight, or 1 to 2% by weight, or 2 to 3% by weight, or 3 to 4% by weight, or 4 to 5% by weight, relative to the total dry matter weight of the fraction.

According to the method of the invention, the fraction enriched in starch and depleted in protein is then mixed with a liquid to form a starch fluid (in other words, a fluid comprising starch). The liquid is preferably water. The starch fluid is preferably in the form of a dispersion, more preferably it is a starch milk. By “starch milk”, is meant, a suspension of starch in water (said suspension may comprise other components, solubilized in water or not).

Preferably, the starch fluid (preferably the starch milk) comprises a dry matter content of 10 to 50% by weight, preferably 20 to 40% by weight, more preferably 25 to 35% by weight, for example, 10 to 15%, or 15 to 20% by weight, or 20 to 25% by weight, or 25 to 30% by weight, or 30 to 35% by weight, or 35 to 40% by weight, or 40 to 45% by weight, or 45 to 50% by weight.

Very advantageously, at least one enzyme is introduced into the starch fluid (preferably the starch milk). The enzyme is preferably a saccharidase, and more particularly an α-amylase. Very preferably, the enzyme is thermostable, particularly at temperatures of 90 to 130° C. The amount of enzyme added is preferably 0.2 to 0.8 kg of enzyme per ton of starch dry matter, preferably 0.3 to 0.5 kg of enzyme per ton of starch dry matter. The pH of the starch fluid is preferably adjusted to a pH between 3.5 and 6.5, more particularly between 4.0 and 6.0. This pH range allows optimal enzyme efficiency. The α-amylases are enzymes capable of hydrolyzing starch into dextrin. However, at this stage of the process, the α-amylase does not have access to the starch molecules that are enclosed in granules.

The starch fluid (preferably the starch milk) is then subjected to a step of hydrolysis of the starch. This step aims to cause the bursting of the starch granules in order to release the starch molecules into the fluid, so as to allow the action of the enzymes (this step aiming to cause the bursting of the starch granules can also be called “liquefaction”). The bursting of the starch granules is achieved by heating the starch fluid (preferably the starch milk) to a temperature allowing the introduction of liquid into the granule, causing the granule to swell and then burst. A hydrolyzed starch fluid is then obtained.

According to the invention, hydrolysis is performed by mixing steam with the starch fluid, preferably using a direct steam injection device.

Preferably, the starch fluid is heated (by mixing with steam) to a temperature (referred to in the present text as the hydrolysis temperature or liquefaction temperature) ranging from 90 to 130° C., preferably from 90 to 100° C.

In a particularly advantageous manner, hydrolysis is performed by mixing a steam flow with a starch fluid flow. By “flow”, is meant a fluid (gas or liquid) in movement.

The mixing of the flows is more preferably operated continuously, in other words, the introduction of at least one fluid to be mixed, and preferably both fluids, into the mixer is performed at least partially simultaneously with the discharge of said mixture from the mixer.

The mixing of steam with the starch fluid in the form of flows allows a very rapid, even quasi-instantaneous, temperature rise of the starch fluid. Relative to the use of tanks (of generally 300 to 500 m³, for a residence time of 1 to 2 h) equipped with steam injection rods that are traditionally used, the mixing of fluids in the form of flows in continuous mode allows faster heating, reduced steam consumption, and reduced energy consumption. It is estimated that greenhouse gas emissions can be reduced by about 40%. Consequently, the implementation of the step of hydrolysis by continuous mixing of steam with the starch fluid in the form of flows allows an even greater reduction in the greenhouse gas emissions of the method.

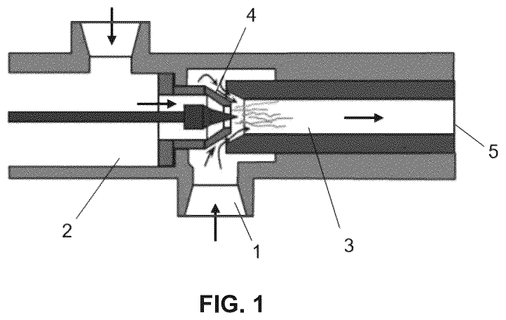

In a particularly preferable manner, hydrolysis is performed using a device for direct steam injection in continuous mode, more preferably a “jet-cooker” device. An example of such a device is shown in FIG. 1.

Preferably, the device for direct steam injection in continuous mode comprises a starch fluid supply line 1 to introduce the fluid into a tube 3 called a “mixing tube”, and a steam supply line 2 ending with a steam injector 4 to inject steam into the mixing tube 3. Preferably, the steam and starch fluid injection points in the mixing tube 3 are arranged according to a coaxial arrangement. Advantageously, the steam injector 4 forms a needle in the starch fluid supply line 1. The starch fluid and the steam flows are mixed within the mixing tube 3. The mixing tube 3 comprises a fluid outlet 5, connected to an outlet pipe allowing the discharge of the mixture. The starch fluid and steam flows can be adjusted independently of each other, for example, using valves.

Preferably, the pressure of the starch fluid injected into the mixing tube is 0.2 to 1 MPa, more preferably 0.4 to 0.7 MPa. In particular, the pressure of the starch fluid injected can be 0.2 to 0.4 MPa, or 0.4 to 0.5 MPa, or 0.5 to 0.6 MPa, or 0.6 to 0.7 MPa, or 0.7 to 0.8 MPa, or 0.8 to 1 MPa. The pressure of the steam injected into the mixing tube is advantageously 0.4 to 1.5 MPa, more preferably 0.6 to 1 MPa. Notably, the pressure of the steam injected can be 0.4 to 0.6 MPa, or 0.6 to 0.8 MPa, or 0.8 to 1 MPa, or 1 to 1.2 MPa, or 1.2 to 1.5 MPa. The pressure of the mixture at the outlet of the mixing tube is preferably 0.1 to 0.5 MPa, more preferably 0.2 to 0.4 MPa. In particular, the pressure of the mixture at the outlet of the mixing tube can be 0.1 to 0.2 MPa, or 0.2 to 0.3 MPa, or 0.3 to 0.4 MPa, or 0.4 to 0.5 MPa. Preferably, the pressure difference between the pressure of the mixture at the outlet of the mixing tube and the pressure of the starch fluid injected into the mixing tube is 0.1 to 0.5 MPa, preferably 0.2 to 0.4 MPa. This pressure difference can, for example, be 0.1 to 0.2 MPa, or 0.2 to 0.3 MPa, or 0.3 to 0.4 MPa, or 0.4 to 0.5 MPa. Such pressure ranges allow optimal homogenization of the steam/starch fluid mixture.

Preferably, the outlet pipe has the following geometry:
a nominal diameter DN over a pipe length of 50 to 200 mm after the fluid outlet, then
a diameter D1 equal to 1.5 to 2.5 times the nominal diameter DN over a pipe length equal to 10 to 30 times the nominal diameter DN, then
a diameter D2 equal to 2 to 5 times the nominal diameter DN.

Such a geometry allows to control the increase in viscosity caused by the swelling of the granules that occurs before their bursting and thus to limit the risks of damage and explosion of the device.

The hydrolyzed starch fluid, preferably not cooled, can then be introduced into a tank. The residence time of the fluid in the tank is preferably 1 to 4 h. This step, called dextrinization, allows the enzyme contained in the fluid to continue hydrolyzing the starch. Advantageously, the dextrinization is carried out until a dextrose equivalent (DE) of 12 to 14 is obtained. The DE is an indicator of starch hydrolysis. When DE=0, the starch is intact. When DE=100, the starch is completely converted into glucose. The method used for DE measurement is the Lane-Eynon method.

The hydrolyzed starch fluid can be used as a fermentation substrate, optionally after one or more additional treatments.

The method according to the invention can, in particular, comprise a step of introducing at least one enzyme into the hydrolyzed starch fluid. This step is called “saccharification” and enables the hydrolysis of dextrin into glucose. In the present text, the term “saccharification” is used to refer to any process of hydrolyzing dextrin into glucose, regardless of the degree of hydrolysis achieved; the step of saccharification can also be called “pre-saccharification” when hydrolysis is not complete or nearly complete.

Preferably, prior to the introduction of enzymes, the hydrolyzed starch fluid is introduced into a tank.

The enzyme(s) introduced are preferably chosen from the group consisting of glucosidases. More preferably, at least 2 enzymes are introduced into the hydrolyzed starch fluid, more preferably at least one α-1,4-glucosidase and one amylo-α-1,6-glucosidase are introduced into the hydrolyzed starch fluid. The α-1,4-glucosidase hydrolyzes the α-(1,4) bonds involved in the linear glucose chains of dextrin; the enzyme amylo-α-1,6-glucosidase (also called “debranching enzyme”) hydrolyzes the bonds involved in the branching of the chains. Advantageously, the pH is adjusted to a value of 3.5 to 5.0, preferably 4.0 to 4.5. The temperature of the medium is preferably set between 50 to 70° C., preferably between 55 to 65° C. These conditions allow optimal enzyme functioning. The amount of enzymes introduced can be 0.2 to 0.6 kg per ton of starch dry matter.

In some embodiments, the duration of saccharification is 2 to 6 h, preferably 3 to 4 h (in these embodiments, this step is more particularly referred to as “pre-saccharification”).

In other embodiments, the duration of saccharification is 30 to 70 h, preferably 40 to 60 h. Advantageously, the amount of glucose in the medium after such a step of saccharification is 90 to 99% by weight, preferably 92 to 97% by weight. The amount of glucose is determined according to the NF EN ISO 10504 standard.

A glucose-enriched medium is obtained, usable as a fermentation substrate, as it is or after optional additional treatments, for example, purification treatments, particularly filtration and/or demineralization treatments. By “glucose-enriched medium”, is meant a medium in which the glucose concentration is higher than that of the hydrolyzed fluid before saccharification.

Preferably, when the substrate undergoes a pre-saccharification step of 2 to 6 h, it is not subjected to further purification.

Preferably, when the substrate undergoes a step of saccharification of 30 to 70 h, it undergoes at least one subsequent step of purification, preferably a filtration and a demineralization, preferably using ion exchange resins.

Advantageously, the method for preparing a fermentation substrate according to the invention generates a greenhouse gas emission of less than 100 kg CO₂ oil equivalent (CO₂ eq) per ton of leguminous plant seed used, preferably less than 60 kg of CO₂ eq per ton of leguminous plant seed used, more preferably less than 40 kg of CO₂ eq per ton of leguminous plan seed used (for example, the method for preparing a fermentation substrate according to the invention can generate a greenhouse gas emission of 0 to 20, or 20 to 30, or 30 to 40, or 40 to 50, or 50 to 60, or 60 to 80, or 80 to 100, kg of CO₂ eq per ton of leguminous plant seed used). Greenhouse gas emissions can be determined as indicated in the Examples section below.

The invention also relates to a fermentation substrate obtained by, or able to be obtained by, a preparation method as described above.

The fermentation substrate according to the invention advantageously comprises one or more of the following features (particularly when obtained by a method including a step of pre-saccharification):
an amount of protein, relative to the total dry matter weight of the substrate, of 5 to 25% by weight, preferably 15 to 20% by weight, for example, 5 to 10% by weight, or 10 to 15% by weight, or 15 to 20% by weight, or 20 to 25% by weight;
an amount of carbohydrate, relative to the total dry matter weight of the substrate, of 40 to 80% by weight, preferably 60 to 75% by weight, for example, 40 to 50% by weight, or 50 to 60% by weight, or 60 to 70% by weight, or 70 to 80% by weight;
an amount of fat (lipid), relative to the total dry matter weight of the substrate, of 0.5 to 5% by weight, preferably 0.5 to 3% by weight, for example, 0.5 to 1% by weight, or 1 to 2% by weight, or 2 to 3% by weight, or 3 to 4% by weight, or 4 to 5% by weight;
an amount of fiber, relative to the total dry matter weight of the substrate, of 2 to 10% by weight, preferably 3 to 6% by weight, for example, 2 to 4% by weight, or 4 to 6% by weight, or 6 to 8% by weight, or 8 to 10% by weight.

The quantities of protein, carbohydrate, fat, and fiber can be determined as indicated above.

Alternatively, the fermentation substrate according to the invention can advantageously comprise one or more of the following features (particularly when obtained by a method including a step of saccharification of 30 to 70 h):
an amount of carbohydrate, relative to the total dry matter weight of the substrate, greater than or equal to 90% by weight, preferably greater than or equal to 95% by weight;
an amount of protein, relative to the total dry matter weight of the substrate, less than or equal to 500 ppm by weight, preferably less than or equal to 200 ppm by weight, more preferably less than or equal to 100 ppm by weight;
an amount of ash (in other words, all minerals, including NaCl and CaCl₂)), relative to the total dry matter weight of the substrate, less than or equal to 200 ppm by weight, preferably less than or equal to 100 ppm by weight, more preferably less than or equal to 50 ppm by weight.

The invention also relates to a method of fermentation comprising bringing a fermentation substrate such as described above into contact with at least one microorganism. The microorganism is preferably chosen from the group constituted of yeasts, bacteria, and combinations thereof.

In some embodiments, the microorganism is brought into contact with, as the substrate, a glucose-enriched medium (i.e., having undergone the step of saccharification) as described above. For this, preferably, the substrate is introduced into a tank, and the microorganism is added to the tank. More preferably, the substrate has been cooled to a temperature of 20 to 40° C., more preferably 25 to 35° C., even more preferably 26 to 30° C. prior to being brought into contact with the microorganism.

In other embodiments, the microorganism, preferably one or more yeasts, is brought into contact with, as the substrate, a hydrolyzed starch fluid (i.e., not having undergone the saccharification step) as described above. Preferably, the hydrolyzed starch fluid is previously cooled to a temperature of 20 to 40° C., more preferably 25 to 35° C., more preferably 26 to 30° C. Preferably, the substrate is brought into contact with the microorganism and with at least one enzyme, more preferably at least two enzymes, preferably chosen from the group consisting of glucosidases. More particularly preferably, the substrate is brought into contact with the microorganism and with at least one α-1,4-glucosidase and one amylo-α-1,6-glucosidase. This step is called “propagation step.” Advantageously, the substrate is introduced into a tank, and the microorganism and enzymes are added to the tank. The amounts of enzyme and the pH are advantageously as described above in relation to the saccharification step.

The invention also relates to a fermentation method, or a method for producing a
fermentation product, comprising the steps of:
preparing a fermentation substrate according to the method described above; and
bringing into contact the fermentation substrate with at least one microorganism.

The step of putting the fermentation substrate into contact with at least one microorganism can be as described above. It corresponds to a fermentation step.

Bringing the fermentation substrate into contact with the microorganism(s) advantageously results in obtaining a fermentation product, particularly as described below.

The invention also relates to a fermentation product obtained by, or able to be obtained by, a fermentation method, or a method for producing a fermentation product, as described above. Preferably, the fermentation product comprises (or consists of) at least one compound chosen from the group consisting of alcohols, preferably ethanol, organic acids, amino acids, vitamins, and mixtures thereof.

Preferably, when the fermentation product is or comprises a compound chosen from among organic acids, amino acids, vitamins, and mixtures thereof, a substrate, having been prepared by a method comprising a saccharification step of 30 to 70 h, is used in the fermentation method (or in the method for producing a fermentation product).

Preferably, when the fermentation product is or comprises a compound chosen from among alcohols, and is in particular ethanol, a substrate having been prepared by a method comprising a pre-saccharification step is used in the fermentation method (or in the method for producing a fermentation product), or a hydrolyzed starch fluid is used as the substrate (in the latter case, the fermentation method or the method for producing a fermentation product very preferably comprises a propagation step as described above).

### EXAMPLES

The following examples illustrate the invention without limiting it.

A method for producing ethanol by fermentation using a substrate prepared from leguminous plant (peas) according to the invention was compared to a method for producing ethanol by fermentation using a substrate prepared from cereal (wheat) and a method for producing ethanol by fermentation using a substrate prepared from beet.

Method for Producing Ethanol from Beet

Once harvested, the beets are quickly transported and processed. The beets are then washed with water in order to remove all traces of soil, grass, or stones coming from the harvest.

After leaving the washer, the beets are cut into cossettes (which resemble potato chips). The size of the cossettes is 5 to 6 cm long and 2 to 3 mm wide. This cutting is done using a root cutter of the Model 2000-600-60 type from company MAGUIN.

The cossettes are then conveyed to the diffusion, in which they circulate counter-current with hot water at 80° C., in which the soluble compounds of the beet migrate to give a diffusion juice exiting at the head of the diffuser. The cossettes exit at the rear of the diffuser in the form of pulp, and part of the water they contain is removed by pressing or dehydration to be recycled. The pulp can then be used in animal feed.

The diffusion juice is then purified by treatment with quicklime, resulting in the precipitation of some impurities. Quicklime is obtained by calcining limestone at 900° C. at a rate of 4.6 kg of stone per ton of beet. The CO₂ produced during this operation is subsequently used in the diffusion juice to precipitate the slaked lime (Ca(OH)₂) into calcium carbonate. The impurities and precipitated lime are separated from the juice by filtration using a filter press.

The purified juice contains 85% water by weight. It is subjected to evaporation in order to be concentrated until a syrup having a sucrose concentration close to saturation, that is, 60 to 70% by weight, is obtained. Evaporation takes place in a multiple-effect evaporator, the pressure being lowered from effect to effect to reduce the boiling point of the concentrated juice, which prevents its cooking. The concentration of the juice enables sugar fermentation to be prevented and thus enables the juice to be able to be stored before being sent to the distillery workshop.

The fermentation step begins with the preparation of the pre-fermentation. The syrup is diluted to 7% by weight of sugar (sucrose) by adding water and is then introduced into a tank called a pre-fermenter with a solution of Saccharomyces cerevisiae yeasts. The amount of yeast is 6 g per ton of sugar. Urea, at a rate of 1.3 kg per ton of sugar, is added, as well as nitric acid in order to maintain the pH between 5.0 and 5.5. The temperature is adjusted between 3° and 35° C. The residence time is set between 4 and 5 h, and a continuous flow of air is injected into the medium by means of a diffuser installed at the bottom of the tank.

When the alcohol content (notably, ethanol) has reached a value between 6 and 8% by weight (as measured using a densimeter), the so-called weak must is introduced into a fermentation tank. A sugar solution with a concentration between 20 and 25% (strong must) is added in a proportion of 30 to 50% by weight of the weak must. The alcoholic fermentation which is exothermic continues while the temperature is maintained between 3° and 35° C. by means of a plate heat exchanger, which is itself connected to a cooling tower. Fermentation takes place for a duration between 30 and 40 h. During this fermentation, the pH tends to decrease. A sodium hydroxide solution is therefore added over time to maintain the pH between 5.0 and 5.5. When all the sugar is converted into ethanol and carbon dioxide, the alcohol content in the fermenter is between 10 and 14% by weight.

The fermentation must is then subjected to a distillation step consisting of separating the alcohol fraction from the fermentation must. The latter is introduced in the middle of a vacuum distillation column containing several trays to then fall to the bottom of the column. The fluid is then heated to the boiling point of the water and ethanol mixture, at a temperature between 82 and 85° C. by means of a heat exchanger supplied with steam from a boiler. The alcohol vapors are collected at the top of the column with an alcohol degree between 93 and 95% by weight.

The distillate obtained contains many impurities such as volatile compounds and other types of alcohol such as methanol and is therefore purified by passing through the following different columns, which together form the rectification step:
Extraction column: In the column, water is added to the distillate coming from the distillation column. The difference in volatility of the compounds present in the distillate allows them to be separated. The compounds that are very volatile and poorly soluble in water are carried to the top of the column, while ethanol and methanol soluble in water are carried to the bottom of the column.
Rectification column: The alcohol-water mixture coming from the extraction column is again distilled until it almost reaches the azeotropic alcohol/water mixture (97% by weight of alcohol) at the bottom of the column, while the impurities are collected at the top of the column (distillate). The impurities recovered from the previous column are introduced into this distillate.
Demethylation column: This large column containing a very large number of trays allows the separation of methanol and other impurities from ethanol. The product obtained is called “surfin alcohol.” The residue containing the methanol is mixed with the impurities collected from the previous step.
Head column: All the impurities from the previous columns are passed through this column to purify them to produce fusel oils. The ethanol recovered at the bottom of the column is recycled in the extraction column or the rectification column to improve the ethanol purification yield. The fusel oils are collected at the top of the column.

All the rectification operations use the liquid separation method by vaporization-condensation fractionation.

During the distillation step, an insoluble residue is collected with the non-evaporated water. This residue is called vinasse. The dry matter is about 6% by weight. This dry matter being too low, the residue is introduced into a falling film tubular evaporator. Evaporation is carried out under vacuum (0.02 MPa pressure) with steam injection. The final dry matter of the residue is 30 to 35% by weight. The concentrated vinasse can be used in agriculture, particularly for preparing an amendment.

For an ethanol production unit, the amount of CO₂ oil equivalent (CO₂ eq) has been estimated. The amount of CO₂ oil equivalent represents all greenhouse gas emissions (including nitrous oxide, methane, etc., in addition to CO₂). Its calculation is performed using “emission factors” the values of which are obtained from the following databases:
EUROPEAN COMMISSION: Note on the conducting and verifying actual calculations of the GHG emission savings, 2015;
INSTITUT FÜR ENERGIE-UND UMWELTFORSCHUNG HEIDELBERG (IFEU): Biograce. Harmonized calculations of biofuel greenhouse gas emissions in Europe.—www.biograce.net;
BUNDESANSTALT FÜR LANDWIRTSCHAFT UND ERNÄHRUNG (BLE): Leitfaden Nachhaltige Biomasseherstellung, 1st Edition, Bonn, 2010;
IINAS INTERNATIONALES INSTITUT FÜR NACHHALTIGKEITSANALYSEN UND-STRATEGIEN, ÖKO-INSTITUT E. V. INSTITUT FÜR ANGEWANDTE ÖKOLOGIE E.V., Gemis, 2014.

The results for the ethanol production method from a beet-derived substrate described above are presented in the table below. Ethanol production is 88,830 t/year (from 1,189,256 t of beet). The CO₂ eq emitted during sugar extraction is attributed 94% by weight to the production and processing of the sugar juice and 6% by weight to the production of pulp (as indicated under the mention “considered yield” in the table below).

TABLE 1

| | | |
|---|---|---|
| | Emission | |
| | factor | |
| | (kg | kg |
| Amount | C0₂eq/unit) | C0₂eq |
| (A) | (B) | (A × B) |

Agricultural emissions

| Seed (kg/Ha/year) | 6 | 3.54 | 366,828 |
|---|---|---|---|
| Nitrogen fertilizers | 119.7 | 5.88 | 12,155,695 |
| (kg/Ha/year) | | | |
| P₂O₅ fertilizer (kg/Ha/year) | 59.7 | 1.01 | 1,041,268 |
| K₂O fertilizer (kg/Ha/year) | 134.9 | 0.58 | 1,351,289 |
| CaO fertilizer (kg/Ha/year) | 400 | 0.13 | 898,073 |
| Pesticides (kg/Ha/year) | 1.3 | 10.97 | 16,703,745 |
| Nitrous oxide (kg CO₂eq/kg | 119.7 | 8.08 | 16,703,745 |
| N) | | | |
| Diesel (agriculture) | 175.9 | 3.14 | 9,539,021 |
| (L/Ha/year) | | | |
| Diesel (transportation) | 67 | 3.14 | 10,424,820 |
| (L/Ha/year) | | | |
| Subtotal (kg CO₂eq/t | 558 | | |
| ethanol) | | | |
| Yield | | | |
| considered: 94% | | | |

Sugar extraction emissions

| Natural gas (MJ/year) | 442,377,866 | 0.067 | 29,639,317 |
|---|---|---|---|
| Electricity (kW/year) | 17,856,000 | 0.61 | 10,892,160 |
| produced by coal-fired | | | |
| power plant | | | |
| Limestone (kg/year) | 54,985,000 | 0.00972 | 534,454 |
| Water (kg/year) | 507,018,000 | 0.0004 | 202,807 |
| Water treatment (kg/year) | 763,269,000 | 0.00027 | 206,083 |
| Subtotal | 439 | | |
| (kg CO₂eq/t ethanol) | | | |

Ethanol production emissions

| Natural gas (MJ/year) | 731,256,874 | 0.067 | 48,994,211 |
|---|---|---|---|
| Electricity (kW/year) | 30,153,046 | 0.61 | 18,393,358 |
| Nitric acid at 65% by weight | 144,324 | 1.89 | 272,772 |
| (kg/year) | | | |
| Sodium hydroxide at 50% by | 731,600 | 0.47 | 343,852 |
| weight (kg/year) | | | |
| Yeasts (kg/year) | 1,193 | 3.2 | 3,818 |
| Urea (kg/year) | 366,268 | 0.81 | 296,677 |
| Water (kg/year) | 137,514,574 | 0.0004 | 55,006 |
| Water treatment | 212,241,923 | 0.00027 | 57,305 |
| (kg/year) | | | |
| Subtotal | 770 | | |
| (kg CO₂eq/t ethanol) | | | |

Total Process

| Total | 1767 |
|---|---|
| (kg CO₂eq/t ethanol) | |
| Total | 990 |
| (kg CO₂eq/t ethanol) | |
| without the vinasse | |

The total amount of CO₂ eq calculated for the total method is distributed as follows: 56% by weight of the total is allocated to ethanol production, and 44% by weight of the total is allocated to the production of the co-product vinasse. The share of greenhouse gas emissions corresponding to ethanol production only is indicated in the table above on the line “Total (kg CO₂ eq/t ethanol) without the vinasse”.

Method for Producing Ethanol from Wheat.

Wheat grains are harvested. Unlike beet, harvested wheat can be stored and kept for several months. Upon arrival at the industrial site, the wheat is first cleaned in order to remove stones and plant residues from the harvest.

The cleaned wheat is then ground by means of a compression grinder of the roller-mill type, then sieved to separate the bran (wheat hull) from the flour (kernel powder). These steps up to the collection of the flour are carried out dry and at room temperature.

The flour is then suspended in water in such a way as to have a dry matter content between 25 and 35% by mass. The fluid thus obtained is introduced into an 80 m³ tank equipped with steam injection rods. At the same time an alpha-amylase enzyme (Maxamyl HT) is added in an amount between 0.2 and 0.6 kg per ton of flour. The pH is adjusted between 5.0 and 6.5 by adding sulfuric acid. Steam is injected into the fluid using the injection rods until the fluid reaches a temperature between 9° and 95° C. This temperature allows water to enter the starch granules until the protective membrane of the granules bursts. The starch released into the fluid is then hydrolyzed by the enzyme. The residence time is between 4 and 5 h to ensure that the starch is sufficiently cut into dextrin chains.

The hydrolyzed must is sent to a 50 m³ tank under stirring, and the enzyme Deltazyme GA LE-5. containing an alpha 1-4 glucosidase and an alpha 1-6 glucosidase is added to perform saccharification. The residence time in the tank is 2 h.

The following steps (fermentation. distillation, and rectification) are identical to those described above for the ethanol production method from beet.

The amount of CO₂ oil equivalent emitted by the method has been estimated as indicated above and the results are presented in the table below. Ethanol production is 23,887 t/year (from 80,235 t of wheat). The CO₂ eq emitted up to the saccharification step (not included) is attributed 84% to the production and processing of the flour and 16% to the production of fiber (the bran) (as indicated under the mention “considered yield” in the table below).

TABLE 2

| | | |
|---|---|---|
| | Emission | |
| | factor | |
| | (kg | kg |
| Amount | CO₂eq/unit) | CO₂eq |
| (A) | (B) | (A × B) |

Agricultural emissions

| Wheat cultivation (t/year) | 80,235 | 300 | 24,070,500 |
|---|---|---|---|
| Diesel (transport) (L/Ha/year) | 15 | 3.14 | 539,867 |
| Subtotal | 865 | | |
| (kg CO₂eq/t ethanol) | | | |
| Considered yield: 84% | | | |

Fermentation substrate production emissions

| Electricity (kW/year) produced | 4,477,249 | 0.04 | 179,090 |
|---|---|---|---|
| by nuclear power plant | | | |
| Subtotal | 439 | | |
| (kg CO₂eq/t ethanol) | | | |

Ethanol production emissions

| | | | |
|---|---|---|---|
| Natural gas (MJ/year) | 351,254,862 | 0.067 | 23,534,076 |
| Electricity (kW/year) | 12,241,584 | 0.04 | 489,663 |
| Sulfuric acid at 96% by weight | 1,730,122 | 0.21 | 363,326 |
| (kg/year) | | | |
| Sodium hydroxide | 157,204 | 0.47 | 73,886 |
| at 50% by weight (kg/year) | | | |
| Yeasts (kg/year) | 856 | 3.2 | 2,739 |
| Ammonia at 24% by | 12,755 | 2.7 | 234,439 |
| weight (kg/year) | | | |
| Water (kg/year) | 226,800,000 | 0.0004 | 90,720 |
| Water treatment (kg/year) | 239,400,000 | 0.00027 | 64,638 |

| | |
|---|---|
| Subtotal | 1032 |
| (kg CO₂eq/t ethanol) | |

Total Process

| Total | 1904 |
|---|---|
| (kg CO₂eq/t ethanol) | |

| | |
|---|---|
| Total | 1066 |
| (kg CO₂eq/t ethanol) | |
| Without the vinasse | |

The total amount of CO₂ eq calculated for the total method is distributed as follows: 56% by weight of the total is allocated to ethanol production, and 44% by weight of the total is allocated to the production of the co-product vinasse. The share of greenhouse gas emissions corresponding to ethanol production only is indicated in the table above on the line “Total (kg CO₂ eq/t ethanol) without the vinasse.”

Method for Producing Ethanol from Peas.

Once harvested, the peas are dehulled by grinding by means of a pendular mill using compression force, then the produced particles are passed over a sieve to separate the hulls smaller than 500 μm from the hulls larger than or equal to 500 μm.

The selected particles (smaller than 500 μm) are then subjected to a micronization step by means of a grinder using impact force (to achieve a median volume diameter (D50) of the particles less than or equal to 30 μm). At the outlet of the grinder, the obtained flour is introduced into turbo-cyclones with a selector supplied with an air flow allowing a cyclonic effect to be activated, in order to separate the particles according to their density. The light particles (called protein fraction), having a D50 of 1 to 5 μm. are carried to the top of the cyclone, while the heavier particles, having a D50 of 10 to 30 μm, are carried to the bottom.

The heavy fraction (called starch fraction) is recovered and mixed with water to achieve a fluid having a dry matter content of about 30% by mass. The pH is adjusted between 4.0 and 6.0 by sulfuric acid, then an alpha-amylase enzyme (Maxamyl HT) is added to the fluid in an amount of 0.3 to 0.5 kg per ton of starch dry matter.

In order to cause the bursting of the starch granules it contains, the fluid is then heated to 100° C. by direct steam injection in continuous mode in a “jet-cooker” of the Hydra-thermal K510 type, (pressure of the fluid containing the starch granules: 0.6 MPa; steam pressure: 0.9 MPa; pressure of the fluid/steam mixture at the jet-cooker outlet: 0.25 MPa).

The fluid is then subjected to a dextrinization step: for this, the non-cooled fluid is placed in a 30 m³ tank for a duration of 4 h. The fluid, cooled to 60° C., is then introduced into another tank to undergo a pre-saccharification step. An α-1.4-glucosidase and an amylo-α-1.6-glucosidase (Deltazyme GA LE-5) are introduced into the fluid in an amount of 0.3 kg per ton of starch dry matter, and the pH is adjusted to a value between 4.0 and 4.5 by sulfuric acid. The fluid is left in the tank for a duration of 3 h.

The following steps (fermentation. distillation, and rectification) are identical to those described above for the ethanol production method from beet, except for the fact that the distillation column is not heated by a heat exchanger supplied with steam from a boiler but by the vapors of the evaporator used to concentrate the vinasse, recompressed by means of a mechanical compressor. The energy used is, in this case, electricity and not gas.

The amount of CO₂ oil equivalent emitted by the method has been estimated as indicated above, and the results are presented in the table below. Ethanol production is 7,465 t/year (from 32,982 t of wheat). The CO₂ eq emitted up to the pre-saccharification step (not included) is attributed 67.2% by weight to the production and processing of the starch fraction, 24% by weight to the production of the protein fraction, and 8.8% to the production of fiber (hull) (as indicated under the mention “considered yield” in the table below).

TABLE 3

| | | |
|---|---|---|
| | Emission | |
| | factor | |
| | (kg | kg |
| Amount | CO₂eq/unit) | CO₂eq |
| (A) | (B) | (A × B) |

Agricultural emissions

| Pea cultivation | 32,982 | 200 | 6,596,400 |
|---|---|---|---|
| (t/year) | | | |
| Nitrogen fertilizer avoided | 32,982 | −189 | −6,233,598 |
| Diesel (transport) (L/Ha/year) | 6.3 | 3.14 | 163,112 |
| Subtotal (kg CO₂eq/t | 70 | | |
| ethanol) | | | |
| Considered yield of 67.2% | | | |

Fermentation substrate production emissions

| Electricity (kW/year) | 6,523,100 | 0.04 | 260,924 |
|---|---|---|---|
| produced by nuclear power | | | |
| plant | | | |
| Subtotal | 35 | | |
| (kg CO₂eq/t ethanol) | | | |

Ethanol production emissions

| | | | |
|---|---|---|---|
| Natural gas (MJ/year) | 78,659,000 | 0.067 | 5,270,153 |
| Electricity (kW/year) | 4,116,000 | 0.04 | 164,640 |
| Sulfuric acid at 96% by | 39,900 | 0.21 | 8,379 |
| weight (kg/year) | | | |
| Sodium hydroxide | 47,161 | 0.47 | 22,166 |
| at 50% by weight (kg/year) | | | |
| Yeasts (kg/year) | 256 | 3.2 | 819 |
| Ammonia at 24% by weight | 38,068 | 2.7 | 102,784 |
| (kg/year) | | | |
| Water (kg/year) | 67,200,000 | 0.0004 | 26,880 |
| Water treatment (kg/year) | 84,000,000 | 0.00027 | 22,680 |

| | |
|---|---|
| Subtotal | 753 |
| (kg CO₂eq/t ethanol) | |

Total Process

| Total | 858 |
|---|---|
| (kg CO₂eq/t ethanol) | |
| Total | 481 |
| (kg CO₂eq/t ethanol) | |
| without the vinasse | |

The total amount of CO₂ eq calculated for the total method is distributed as follows: 56% by weight of the total is allocated to ethanol production, and 44% by weight of the total is allocated to the production of the co-product vinasse. The share of greenhouse gas emissions corresponding to ethanol production only is indicated in the table above on the line “Total (kg CO₂ eq/t ethanol) without the vinasse.”

The results are summarized in the table below (the shares of emissions attributed to ethanol production only are considered):

TABLE 4

| | kg of CO₂ equivalent emitted per ton of ethanol produced | | |
|---|---|---|---|
| | Wheat method | Beet method | Pea method |
| | (comparative) | (comparative) | (invention) |
| Method up to the | 488 | 559 | 60 |
| distillation | | | |
| (not included) | | | |
| Method from the | 578 | 431 | 421 |
| distillation | | | |
| Total | 1,066 | 990 | 481 |

It can be seen that the ethanol production method according to the invention emits much less greenhouse gas than the comparative methods using a substrate prepared from cereal and beet.

Moreover, relating to the method up to the distillation, the method according to the invention produces very low greenhouse gas emissions.

1. A method for preparing a fermentation substrate, comprising the following steps:
providing at least one leguminous plant seed comprising starch and proteins;
micronizing said at least one seed, so as to obtain a micronized fraction;
purifying the micronized fraction, so as to collect a fraction enriched in starch and depleted in protein;
mixing the fraction enriched in starch and depleted in protein with a liquid, so as to form a starch fluid; and
hydrolyzing the starch by mixing steam with the starch fluid, so as to obtain a hydrolyzed starch fluid.

2. The method of claim 1, further comprising a step of introducing into the hydrolyzed starch fluid at least one enzyme chosen from the group consisting of glucosidases.

3. The method of claim 1, further comprising a step of introducing into the hydrolized starch fluid at least one enzyme chosen from the group consisting of saccharidases.

4. The method of claim 1, wherein the leguminous plant seed comprises a hull, and wherein the method comprises a step of dehulling the seed prior to the micronization.

5. The method of claim 1, wherein the purification of the micronized fraction comprises a step of aeraulic separation.

6. The method of claim 1, wherein the hydrolysis of the starch is performed by means of a device for direct steam injection in continuous mode.

7. The method of claim 1, wherein the leguminous plant is chosen from the group consisting of beans, peas, broad beans, lentils, chickpeas and mixtures thereof.

8. The method of claim 1, which generates a greenhouse gas emission of less than 100 kg CO₂ oil equivalent per ton of leguminous plant seed used.

9. A fermentation substrate obtained by the method of claim 1.

10. A method of fermentation comprising bringing into contact the fermentation substrate of claim 9 with at least one microorganism.

11. A method for producing a fermentation product, comprising the following steps:
preparing a fermentation substrate according to the method of claim 1; and
bringing into contact said fermentation substrate with at least one microorganism, so as to obtain a fermentation product.

12. The method of claim 11, wherein the fermentation product comprises at least one compound chosen from the group consisting of alcohols, organic acids, amino acids, vitamins and mixtures thereof.

13. The method, of claim 11, wherein the microorganism is chosen from the group consisting of yeasts, bacteria and combinations thereof.

14. A fermentation product obtained by the method of claim 11.

15. The fermentation product of claim 14, comprising at least one compound chosen from the group consisting of alcohols, organic acids, amino acids vitamins, and mixtures thereof.

16. The method of claim 1, further comprising a step of introducing into the hydrolyzed starch fluid at least two enzymes chosen from the group consisting of glucosidases.

17. The method of claim 1, further comprising a step of introducing into the hydrolyzed starch fluid at least one α-1.4-glucosidase and one amylo-α-1.6-glucosidase.

18. The method of claim 1, further comprising a step of introducing into the starch fluid an α-amylase.

19. The method of claim 5, wherein the aeraulic separation is carried out by means of a cyclone with a selector.

20. The method of claim 11, wherein the fermentation product comprises ethanol.
